(19) United States
(12) Patent Application Publication
(10) Pub. No.: US 2004/0197876 A1
(43) Pub. Date: Oct. 7, 2004

(54) Recombinant fusion proteins and the trimers thereof

(76) Inventors: Jurg Tschopp, Epalinges (CH); Pascal Schneider, Epalinges (CH)

Correspondence Address: CONNOLLY BOVE LODGE & HUTZ, LLP, P O BOX 2207, WILMINGTON, DE, 19899, US

(21) Appl. No.: 10/477,159

(22) PCT Filed: May 8, 2002
(86) PCT No.: PCT/EP02/05103

(30) Foreign Application Priority Data
May 8, 2001 (DE) 101 22 140.1

(51) Int. Cl. C07H 21/04; C12P 21/04; C07K 16/40; C07K 14/54

(52) U.S. Cl. 435/69.7; 435/320.1; 435/326; 530/351; 530/391.1; 530/399; 536/23.5

(57) ABSTRACT

The invention relates to recombinant fusion proteins having the property of being able to form trimers. Said recombinant fusion proteins comprise at least one component A and at least one component B. Component B. Component B has trimerizing properties and component A has biological properties. The invention also relates to trimers of said recombinant fusion proteins. The invention further relates to the use of said trimers in the production of a medicament or the use thereof for in-vitro diagnosis or in the production of in-vitro diagnosis agent. The invention also relates to DNA sequences coding for said fusion protein and expression vectors and host cells containing said DNA sequences or expression vector.

The present invention relates to recombinant fusion proteins which are capable of forming trimers, the recombinant fusion proteins comprising at least one component A and at least one component B, component B having a trimerizing properties and component A biological properties, and to trimers of these recombinant fusion proteins. The present invention furthermore relates to the use of such trimers for the production of a medicament or the use thereof for in-vitro diagnosis or for the production of an in-vitro diagnostic agent. The present invention also relates to DNA sequences encoding such a fusion protein, and to expression vectors and host cells comprising the DNA sequence or the expression vector.

A large number of proteins exist in nature whose physiological form is that of trimers. Interactions at the surfaces of these proteins which trimerize in solution may result in protein aggregations which occur spontaneously or else in a delayed, for example kinetically delayed, manner owing to the fact that these aggregations are concentration- or medium-dependent. The forces responsible are hydrophobic interactions, hydrogen bonds, covalent bonds, for example disulfide bridges, and/or Coulomb forces.

In addition, however, certain proteins have structural motifs which lead to the formation of specific structure-dependent intermolecular supersecondary structures and thus to protein trimers, plus other multimerization states. The formation of supersecondary structures is based on characteristic amino acid sequences of the proteins forming these trimers. Supersecondary structures which can be mentioned are, for example, what are known as “coiled-coil triple helices”, which bring about the trimerization of proteins by the interactions of characteristic α-helices, which are found in each of the proteins forming the coiled-coil form. The coiled-coil triple helix as the intermolecular “trimerization domain” of proteins is—in terms of structure—a three-stranded superhelix coiled around one another. Such coiled-coil motifs with triple helix character are found in particular in extracellular protein trimers, but very especially in proteins or protein complexes of the connective tissue.

Thus, for example, Beck et al. (J. Mol. Biol. (1996) 256, 909-923) describe a connective tissue protein, known as the cartilage matrix protein (CMP), whose aggregation to a homotrimer is based on the triple helix with coiled-coil pattern which is the result of the aggregation of three complementary helices (each as a polypeptide component). It is the heptad pattern (abcdefg)ₙ which is characteristic of the amino acid sequence of such a triple-helix-forming helix. The amino acids of the heptad pattern in positions a and d usually have attached to them apolar side chains, thus permitting the formation of the above-described superhelical structure, in this case a triple helix composed of three helices.

Specific structure-dependent multimerization phenomena which are due to the formation of supersecondary structures are also found in proteins from the collagen family. Here, the structure of collagen fibers is characterized by tropocollagen, which consists of three helical twisted polypeptides. Also, the protofibril of a hair consists of an α-keratin triple helix with the “coiled-coil” motif, albeit in left-handed form.

In addition, the proteins C1q, collagen α1 (X), α2 (VII), the overwintering protein, ACRP30, the inner ear structure protein, cerebellin and multimerin are classed as the protein family under the term C1q family, owing to their sequence homologies in their respective multimerizing sequence segments (Kischore and Reid, Immunopharmacol., 42 (1999) 15-21), and, owing to their structure, these proteins are in the form of higher aggregates of, for example, trimers. Among the proteins with multimerization properties which are found in this family, for example the structure of the protein C1q, which is known from the complement system, is characterized by monomers, each of which has a globular domains which is known as the “head” and a “collagenaceous” helical sequence segment. It is this helical sequence segment, which forms a coiled-coil triple helix, via which the monomers trimerize. In turn, six of these C1q trimers form an oligomer, the oligomerization of the protein trimers, in turn, being based on interactions between the individual coiled-coil triple helices. The result of this structural arrangement of the protein or the multi-(oligo-)merized protein complex C1q is a construction also termed “bouquet”, it being ensured that 18 globular, C-terminally arranged “head” domains are connected to give a hexamer of trimers.

A structure similar to that of the C1q protein is also found in the protein ACRP30, another protein from the C1q family (Hu et al., J. Biol. Chem., Vol. 271, No. 18, 10697-10703, 1996). This serum protein, which is secreted by adipocytes, is most probably quatromers of trimers where globular C-terminal domains are linked via collagenaceous triple helices, as is also the case in the C1q protein. It is assumed that four of these triple helices, in turn, finally form an oligomer by means of suitable interactions. The publication of Shapiro and Scherer (Current Biology 1998, 8:335-338) shows the structure of an ACRP30 homotrimer as determined with the aid of x-ray structure analysis.

Other proteins which are known from the literature are those from the collectin class, which are characterized by a collagenaceous domain, a neck region and in addition by a globular carboxy-terminal lectin binding domain. The collectins too are found physiologically as oligomers of trimers. Thus, for example, the proteins lung surfactant protein A (SP-A) and the mannose binding protein (MBP), each from the collectin family, trimerize owing to the interaction of their “collagenaceous” domain and eventually occur as hexamers of trimers (Epstein et al., Current Opinion in Immunology, Vol. 8, No. 1, 1996, 29-35). Accordingly, the proteins known under the name collectins therefore also form oligomers (for example hexamers) of multimers (for example trimers).

The literature furthermore discloses that a large number of proteins which act physiologically as signal molecules are capable of transducing a biological signal only in specific states. Thus, for example, membrane-bound FasL is biologically, i.e. apoptotically, active while after elimination of the extracellular protein segment from the membrane-bound segment (known as sFasL) said non-membrane-bound sFasL fraction is no longer capable of physiologically acting apoptotically on target cells. The publication of Schneider et al. (J. Exp. Med., Vol. 187, No. 8, 1998, 1205-1213) described how the biological action of sFasL trimers which—as explained above—are obtained after elimination from the membrane-bound protein segment can, however, be reactivated with regard to the physiological function by using crosslinking antibodies. To this end, a fusion protein consisting of the trimerization domain of FasL, a short linker sequence and a flag tag (with the flag amino acid sequence (one-letter code) DYKDDDDK) was constructed and expressed, and such fusion proteins which are non-structure-dependently trimerized (i.e. not via specific secondary-structure interactions with the result that a supersecondary structure is formed) are crosslinked by means of antibodies directed against the flag tag.

The unpublished German patent application DE 19963859 discloses bi- or oligomers of di-, tri-, tetra- or pentamers, that is to say higher-order aggregates, which consist of recombinant fusion proteins encompassing two components A and B. In order for example to increase the biological (apoptotic) activity of TNF cytokines, component A of the recombinant fusion proteins may, in accordance with DE 19963859, for example be a TNF cytokine and component B a protein segment which links the recombinant fusion proteins to give higher-order aggregates.

Although such complexes with potent apoptotic activity are desired in a large number of medical indications, the treatment of a large number of diseases, however, requires the provision of substances which reliably block the triggering of apoptotic events. It is known from the publication of Suda et al. (J. Exp. Med. 1997, 186, pp. 2045-2050) that soluble FasL trimers can under certain circumstances block apoptosis which is induced by an oligomerized molecules. Substances, for example protein-based substances, which are capable of reliably blocking the triggering of, for example, apoptotic events at the receptor itself, which are non-native in nature and which are therefore better protected against physiological degradation in vivo are, however, not known from the prior art.

The object of the present invention is therefore the provision of those substances which, being biomolecules, are capable of acting as a biological block at the receptor itself so that for example triggering of the apoptotic signal transduction cascade is suppressed.

The present object is defined by the subject matter of claim 1, viz. by trimers of recombinant fusion proteins which comprise at least one component A and at least one component B, component A comprising a protein or a protein segment with a biological function, in particular with a binding function, and component B comprising a protein or a protein segment which trimerizes the recombinant fusion proteins without the activity of third molecules, i.e. which generates a trimer of biologically active components A. The invention therefore provides trimers which cannot form higher-order aggregates, for example dimers of trimers, but which, rather, are essentially, at least to 90%, preferably to at least 95% and very especially preferably to at least 99%, in each case based on the total number of trimers, present in solution as trimerized recombinant fusion proteins.

A protein or protein segment with a biological function (component A in the fusion protein) is understood as meaning in particular proteins which have a ligand function, very particularly for antibodies or receptors, (that is to say which are capable of interacting as binding partner with one or more molecule(s)), modified amino acid sequences, for example amino acid sequences with covalently or non-covalently coupled active ingredients (if appropriate of organochemical nature), antibodies or antibody segments with paratopes, or else hormones, for example peptide hormones. In this context, the present invention is based on the finding that in particular signal proteins which, or whose segments or derivatives, are employed as component A in accordance with the invention are biologically active only in the form of higher-order aggregates; in contrast, as trimers, they bind in vitro and in vivo to receptors, but do not activate these receptors but, rather, occupy the binding sites competitively and are not capable of triggering a biologically activating signal, but only of blocking.

Among physiologically membrane-bound signal proteins, for example in the case of TNF cytokines, cleavage products encompassing the extramembranous, in particular the extracellular, protein segments are preferred as component A of a trimerizing recombinant signal protein. However, amino acid sequences which can act as antigens may also be employed as component A in the recombinant fusion protein. Finally, receptors, for example receptors from the family of the TNF receptors (for example FasR) or segments or derivatives of such receptors, which likewise have a binding function (and thus interact as binding partner with another molecule, for example membrane-bound FasL) and which are thus also covered by the term “ligand” for the purposes of the present invention may also be used as component A. Such biological receptor fragments which are capable of binding are particularly suitable for use as medicament when the complementary biological ligand is present in unphysiologically high concentrations in the patient.

In a preferred embodiment, the components A which are present in trimers according to the invention may encompass identical components A (homotrimers) or different components A (heterotrimers), i.e. various recombinant fusion proteins may form a trimer according to the invention. In this manner, proteins with various components A, if appropriate with different biological functions, may be bound together in the trimer according to the invention. Here, the components A of two recombinant proteins may be identical while the third fusion protein may deviate with regard to its component A, or else all three fusion proteins may differ with regard to component A. In this manner, the selection, the arrangement, the specific combination and/or the number of components A in the trimer can typically finely modulated inhibitory effects, if appropriate in combination with activating effects, can be achieved.

In a further preferred embodiment, component A in the recombinant fusion protein takes the form of a peptide hormone, a growth factor, a cytokine, an interleukin or a segment of these, preferably a segment capable of binding. However, functional derivatives of the abovementioned peptides, protein segments and/or proteins may also be employed as component A in the recombinant fusion protein which is a constituent of a trimer according to the invention.

In accordance with a further preferred embodiment of the trimeric recombinant fusion protein according to the invention, its component A encompasses a receptor, for example a receptor for a peptide hormone, a growth factor, a cytokine, an interleukin, or the components A of the fusion protein according to the invention take the form of a segment or a derivative of such a receptor. Especially preferred examples of such a receptor are receptors from the family of the TNF receptors, in particular FasR (hereinbelow also simply referred to as Fas).

The term functional derivatives of biologically active proteins, protein segments or peptides refers in particular to those proteins which maintain the biological function, in particular the binding property with the interaction partner, for example the membrane-bound receptor, but whose sequence shows differences to the corresponding native sequences. These sequence deviations may take the form of one or more insertion(s), deletion(s) and/or substitution(s), a sequence homology of at least 70% being preferred and a sequence homology of at least 85% between the derivative employed and the native sequence being more preferred and at least 90% being very especially preferred. The term functional derivatives covers in particular those amino acid sequences with conservative substitution in comparison with the physiological sequences. The term conservative substitutions refers to those substitutions where amino acids from the same class are exchanged for one another. There are, in particular, amino acids with aliphatic side chains, positively or negatively charged side chains, aromatic groups in the side chains or amino acids whose side chains are capable of forming hydrogen bridges, for example side chains with a hydroxy function. This means that for example an amino acid with a polar side chain is replaced by another amino acid with a likewise polar side chain, or, for example, that an amino acid which is characterized by a hydrophobic side chain is replaced by another amino acid with a likewise hydrophobic side chain (for example serine (threonine) by threonine (serine), or leucine (isoleucine) by isoleucine (leucine)). Insertions and substitutions are possible in particular at those sequence positions which do not bring about a change in the spatial structure or which relate to the binding region. A change of a spatial structure by insertion(s) or deletion(s) can be detected readily with the aid of, for example, CD spectra (circular dichroism spectra) (Urry, 1985, Absorption, circular Dichroism and ORD of Polypeptides, in: Modern Physical Methods in Biochemistry, Neuberger et al. (Ed.), Elsevier, Amsterdam). Suitable methods for generating proteins with amino acid sequences which contain substitutions in comparison with the native sequence(s) are disclosed for example in the publications U.S. Pat. No. 4,737,462, U.S. Pat. No. 4,588,585, U.S. Pat. No. 4,959,314, U.S. Pat. No. 5,116,943, U.S. Pat. No. 4,879,111 and U.S. Pat. No. 5,017,691. The generation of derivatives is described in particular also by Sambrook et al, (1989, Molecular Cloning: A Laboratory Manual, Cold Spring Harbor Laboratory Press), it being possible to leave out, complement or substitute codons. Other derivatives may be in particular those proteins which are stabilized in order to avoid physiological degradation, for example by stabilizing the protein backbone by a substitution of by stabilizing the protein backbone by substitution of the amide-type bond, for example also by employing β-amino acids.

For the purposes of the present invention, a ligand is understood as meaning all molecules which participate in binding reactions. Accordingly, a ligand may also be a protein normally referred to as a receptor. Also, such a receptor may be “ligand” for the purposes of the present invention, for example when it binds to its interaction partner, for example a signal molecule.

A trimer of recombinant fusion proteins is especially preferred when component A in the recombinant fusion protein is a cytokine from the TNF cytokine family, a segment of such a TNF cytokine or a functional derivative of a TNF cytokine or of a corresponding TNF cytokine segment. By binding to the corresponding receptors in vivo (for example in the case of binding in the form of a higher-order aggregate), the biological action of the TNF cytokines employed in the target cells may bring about for example apoptotic, proliferative or activating effects, but then as the trimer typically only ensure the binding to the receptor in question, but no longer exert the activating function. In a nonlimiting enumeration, suitable TNF cytokines, and thus a suitable component A in the fusion protein, are, in particular, the proteins OX40L, RANKL, TWEAK, Lta, Ltab2, LIGHT, CD27L, 41-BB, GITRL, APRIL, VEGI and BAFF or their segments or derivatives. Very especially preferred are the proteins CD40L, FasL, TRAIL, TNF (in particular TNF which binds to the receptor TNF-R2), CD30L and EDA, or their segments or derivatives. A component A in the recombinant fusion protein which is preferably used are extracellular segments of the abovementioned membrane-bound TNF cytokines or their functional derivatives. These cleavage products are very especially preferred when in particular their binding capacity for the receptor in question is retained. Functional derivatives in the abovementioned sense of the abovementioned TNF cytokines or segments of the TNF cytokines may also be employed as component A of the fusion protein. In a very especially preferred embodiment, component A of the recombinant fusion protein, which is a constituent of the trimer according to the invention, is selected from the group consisting of hFasL (AA 139-281), hTRAIL (AA9 5-281), hCD40L (AA 116-261) and m or hTNFα (AA 77-235).

Accordingly, the following possibilities result in accordance with the invention: component A selected for a recombinant fusion protein, which is to become constituent of an oligomer according to the invention, is already in solution in the form of a trimer. In such a case, component B will enhance the trimerization of the components A even more. This situation is found for example when component A, for example a TNF ligand or a segment or derivative thereof, which is typically already trimerized in solution, is to be stabilized in its trimeric form even further by the component B. In contrast, in the event that component A of a recombinant fusion protein as such shows no surface-interaction-mediated trimeric structure in solution or in vivo, component B will, in accordance with the invention, have to ensure trimerization of component A of the recombinant fusion proteins. The latter is the case for example typically when only segments of the native protein, which, as such, cannot trimerize or at least are not present in vivo in trimeric form, for example because the equilibrium is shifted greatly toward the monomer, that is to say, for example, segments of cytokines, in particular C-terminal segments (for example segments encompassing at least 100 AA (in each case calculated from the C terminus), preferably at least 120 AA and particularly preferably at least 150 AA from the C terminus) of FasL, CD40L, CD30L, TRAIL, EDA or TNF, are used as component A of the recombinant fusion protein.

In a preferred embodiment, however, component A of the recombinant fusion protein may also take the form of an amino acid sequence according to the present invention, which is suitable for acting as carrier for a receptor agonist or receptor antagonist. Thus, for example, a pharmacologically active, small organochemical molecule can be coupled, typically covalently, to such an amino acid sequence, for example via an ether bond with threonine or serine, an amide-like bond or via an ester bond. Such coupled agonists or antagonists can increase the binding constant of a trimer according to the invention, preferably to values of at least 10⁻⁹ M⁻¹, or can modulate the biological activity, in particular the inhibitory behavior of a trimer according to the invention, in particular with regard to inhibiting the triggering of the apoptotic signal cascade. In addition, a trimer according to the invention can be employed as carrier for pharmacologically active substances. By selecting a suitable carrier trimer according to the invention, a pharmacologically active ingredient can in this way be transported selectively into the spatial vicinity of specific cells, which constitute the pharmacological targets of these active ingredients. One possibility consists, for example, in coupling such an active ingredient to an FasL trimer, the binding of the FasL trimer blocking the FasR (and thus inhibiting, in accordance with the invention, apoptosis, in this way safeguarding the survival of the cell), while the active ingredient is applied directly to the target cell. A possible use of such a system could result for example from linking the trimer to cytotoxic substances as active ingredient, which substances prevent an attack of immune cells against the target cells, for example in the case of degenerative, in particular neurodegenerative, diseases, especially Parkinson's disease or Alzheimer's disease. In the case of Parkinson's disease, perishing of the dopamine-producing cells in the substantia nigra can thus be prevented in accordance with the invention. The use of such systems of carrier in accordance with the invention and, if appropriate, covalently coupled active ingredient component as medicament in human or veterinary medicine is thus disclosed in general.

Component B of the recombinant fusion protein will typically take the form of a protein from the C1q protein family or the collectin family. Especially preferred are the proteins from the C1q or the collectin family as constituent of the recombinant fusion protein, viz. as component B, when only the trimerization domain, but not the oligomerization domain, is transcribed, or translated, as constituent of the recombinant fusion protein. Preferably, component B in the recombinant fusion protein will also not comprise the globular “head” domain, which is characteristic of the abovementioned proteins in the native state. The abovementioned component B in a recombinant fusion protein according to the invention will thus have a sequence which typically only comprises the, for example collagenaceous, segment, which has the functionality for trimerization due to the formation of a triple helix, but not those sequence segments which additionally have the ability to form a bi- or oligomeric structure with other triple helices (for example a tetra- or hexamer of, for example, triple helices).

The trimerizing fusion protein will therefore typically only contain those domains of the proteins from the C1q protein family or the collectin family as component B which are responsible for the trimerization, while their respective “head” domains will be replaced by other proteins or protein segments as component A which likewise exert a biological function. For the purposes of the present invention, the term “recombinant fusion protein” is thus understood as meaning that the at least one component A and the at least one component B in the recombinant fusion protein are fused artificially, i.e. that a fusion protein for the purposes of the present invention corresponds to no naturally occurring protein.

Functional, i.e. trimerizing, derivatives of proteins of the C1q protein family or the collectin family, or functional derivatives of segments of the abovementioned proteins, may also be employed as component B for the aggregation of recombinant fusion proteins to give trimers. For example, component B will comprise the relevant sequence segments of the proteins C1q, MBP, SP-A (lung surfactant protein A), SP-D (lung surfactant protein D), BC (bovine serum conglutinin), CL43 (bovine collectin-43) and/or ACRP30 or else of functional derivatives of these protein segments.

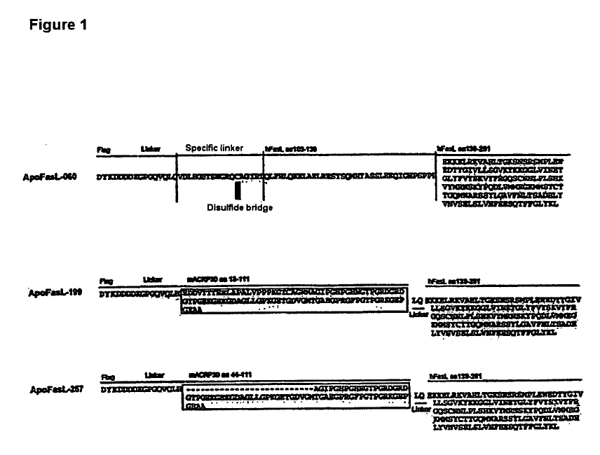

Especially preferred are trimers of recombinant fusion proteins when component B of the recombinant fusion protein comprises a protein segment of the protein C1q or of the protein ACRP30 with a sequence segment of at least 8 AA in length, typically of at least 20 AA in length, from the collagenaceous sequence regions which form a triple helix, or a functional derivative of these. A very especially preferred embodiment of the present invention are trimers of recombinant fusion proteins whose component B comprises an amino acid sequence as shown in FIG. 1 (framed sequence, AA 45 to 111) or a functional derivative of this murine (m) amino acid sequence (for example the analogous human sequence or the analogous sequence of another mammal) or a segment of this sequence.

Especially preferred are trimers of those fusion proteins which comprise sequences from different host organisms. Very especially preferred are aggregates according to the invention when they originate from chimeric fusion proteins, component A originating from a different animal species than component B. Thus, it may be advantageous that component A corresponds to an amino acid sequence from mouse, rat, pig or another vertebrate, in particular a mammal, or to a functional derivative of such a sequence, and component B is of human origin, or vice versa. Alternatively, the sequences of component A and component B in a fusion protein according to the invention which forms a trimer according to the invention may preferably also originate from the same animal species.

In a further preferred embodiment of the present invention, the recombinant fusion proteins trimerize due to a short amino acid sequence of more than 6 amino acids, preferably of 8 to 30, very especially preferably 8 to 20, amino acids, which sequence is present in the recombinant fusion proteins as component B. This trimerization of fusion proteins, which is achieved owing to these short amino acid sequences, is typically based on the formation of supersecondary structures, in particular on the formation of coiled-coil triple helices. Suitable for this purpose are, for example, all those sequence segments of proteins which generate trimers owing to the formation of supersecondary structures, for example typical collagenaceous triple helices or segments of these (as they [lacuna] for example in the case of the proteins CMP, COMP, collagen or laminin).

Since, according to the invention, the component B of the recombinant fusion protein, which leads to the trimerization, should form essentially no higher aggregates, component B should typically not comprise a cysteine residue, which is capable of forming an intermolecular disulfide bridge. Preferably, component B in a recombinant fusion protein will therefore [lacuna] no cysteine residue, or only those cysteine residues which contain an intramolecular disulfide bridge, that is to say within the recombinant fusion protein itself, in order to avoid a situation where a covalent linkage with the at least one cysteine residue of a fusion protein of another trimer may occur under oxidizing conditions.

In addition to components A and B, the fusion protein may comprise additional sequence segments. Sequences which are preferred for the purposes of the present invention are, in this context, those known as tag sequences, for example at least one flag tag, that is to say the amino acid sequence DYKDDDDK, and/or else for example at least one His tag (comprising several consecutive histidines, for example at least five) and/or further tag sequences or antigenic sequences. In addition, the individual segments (components A, B or tag sequences, it also being possible for two or more components A to be present in the fusion protein according to the invention) of a fusion protein according to the invention may be separated from one another by linker sequences. These linker sequences (at least 2 AA, preferably at least 5 AA) serve for the structural delimitation of the various functional components in the recombinant fusion protein and can preferably also exert a “hinge” function, i.e. an amino acid sequence of flexible structure. Very especially preferred are those linkers which comprise at least one proteolytic cleavage site, which enables the components A to be separated from the components B. The proteolytic cleavage site in the linker is preferably a thrombin consensus sequence.

In principle, component A can be arranged C- or N-terminally relative to component B, preferably C-terminally. The tag sequences may occur at any position of a recombinant fusion protein according to the invention, preferably at the N terminus.

Processes for blocking cellular extramembranous receptors are also disclosed as a further subject matter of the present invention. Such processes are characterized by the recombination of at least one component A, which corresponds to a protein or protein segment with a biological function, and at least one trimerizing component B, wherein first (a) such a recombinant fusion protein is expressed, for example in an expression vector, (b) isolated, and then (c) added to a cell culture, for example a cell suspension, for in-vitro studies. The present process is thus suitable for protecting cells, for example from apoptotic cell death, in in-vitro studies. Such cells with trimers according to the invention which are bound as described in the process can then be employed for further in-vitro studies or else for the production of a medicament. If the binding constant is high, such in-vitro treated cells can be retransplanted. For example, such a procedure is suitable in the case of autoimmune diseases or degenerative diseases in order to protect the cells from apoptotic death in vivo.

A process of the above type is very especially preferred when component A is a TNF cytokine, a segment of a TNF cytokine or a functional derivative of such a protein or protein segment.

Trimers of the present invention are suitable for the production of a medicament or for the treatment of diseases or disorders for medicinal use, i.e. for use in both human and veterinary medicine, in particular when component A is a signal protein or a segment of such a signal protein or a derivative of the protein or segment. A wide spectrum of diseases or disorders can be treated with the trimers (hetero- or homotrimers) claimed by the invention. They are used in particular when increased extracellular concentrations of the respective physiological ligands or an increase in the number of membrane-bound receptors is or are observed among the symptoms. Examples are increased concentrations of membrane-bound signal molecules, for example TNF cytokines (for example FasL), on the cells themselves, or of soluble signal molecules, for example TNF cytokine which is soluble as the result of protease cleavage. In a nonlimiting enumeration, such trimers according to the invention can be employed for example for the production of a medicament for the treatment of hyperinflammatory disorders, autoimmune diseases, diseases which are based on hyperapoptotic reactions, or degenerative, in particular neurodegenerative, diseases (for example Parkinson's disease), if appropriate also viral infections. Trimers according to the invention are very particularly suitable when the disease requires a treatment which intends to prevent the biological activity of native cytokines, that is to say serves for blocking corresponding cytokine receptors. Examples to be mentioned are: treatment of viral hepatitis (HBV, HCV), alcohol-induced hepatitis diseases, cholestatic hepatitis, Wilson's disease, hepatitis due to nonfunction of the autoimmune system, of rejection following liver transplants, GvHD, TEN (toxic epidermal necrolysis), Hashimoto's thyroiditis or multiple sclerosis.

The present invention furthermore relates to DNA sequences which encode fusion proteins of the abovementioned type. Such DNA sequences are expressed in expression vectors, the corresponding expression vectors, which comprise a DNA sequence for the fusion proteins according to the invention, also being the subject matter of the invention. The present invention furthermore extends to those host cells which are transfected with DNA sequences which encode the fusion proteins according to the invention. Very especially preferred in this context are host cells which are transfected with expression vectors, the expression vectors, in turn, comprising DNA sequences which encode the fusion proteins according to the invention. All of the abovementioned subjects according to the present invention are suitable as medicaments or for the production of a medicament, in particular for the treatment of diseases which are disclosed in the present patent application, if appropriate as constituent of a composition.

Within the scope of the present invention, the trimers according to the invention, or the other subjects of the present invention, are preferably used in such a way for the production of a medicament or for the treatment of the abovementioned diseases or disorders that they are suitable for parenteral, i.e. for example subcutaneous, intramuscular, intraarterial or intravenous, or oral or intranasal or anal, intraperitoneal, vaginal or buccal, intracerebral, intraocular administration (injection or infusion), if appropriate also for topical application.

Each of the trimers according to the invention or host cells which form trimers according to the invention, or the DNA sequences which encode recombinant fusion proteins capable of forming trimers, or suitable expression vectors, can act as medicament per se or be used for the production of a medicament. However, they may also be employed as medicament in combination with other active ingredient components or pharmaceutical adjuvants, carriers or additives. Thus, the trimers according to the invention or the further subjects of the invention can be combined as constituents in combination with pharmaceutically acceptable carriers, adjuvants and/or additives. Also disclosed in accordance with the present invention are therefore (pharmaceutical) compositions comprising subjects according to the invention, in particular trimers according to the invention. Suitable preparation procedures are disclosed in “Remington's Pharmaceutical Sciences” (Mack Pub. Co., Easton, Pa., 1980), whose contents are herewith incorporated by reference. Suitable carriers for parenteral administration are, for example, sterile water, sterile salines, polyalkylene glycols, hydrogenated naphthalene and, in particular, biocompatible lactide polymers, lactide/glycolide copolymer or polyoxyethylene/polyoxy-propylene copolymers. Compositions according to the invention may comprise fillers or substances such as lactose, mannitol, substances for covalently linking polymers, such as, for example, polyethylene glycol, to inhibitors according to the invention, complexing with metal ions or inclusion of materials in or on specific preparations of polymer compound, such as, for example, polylactate, polyglycolic acid, hydrogel, or on liposomes, microemulsion, micelles, unilamellar or multilamellar vesicles, erythrocyte fragments or spheroplasts. The choice of the individual embodiments of the compositions depends on the physical behavior, for example with regard to solubility, stability, bioavailability or degradability. Controlled or constant release of the active ingredient component according to the invention in the composition includes formulations based on lipophilic depots (for example fatty acids, waxes or oils). Coatings of substances according to the invention or compositions comprising such substances, viz. coatings with polymers, are likewise disclosed within the scope of the present invention (for example poloxamers or poloxamines). Furthermore, substances or compositions according to the invention may be provided with protective coatings, for example protease inhibitors or permeabilizing agents.

Trimers according to the invention are preferably also used in the field of in-vitro diagnosis or else for example for biochemical purification processes. The use of trimers according to the invention in the context of biochemical purification processes, in particular chromatographic processes, for example on purification columns which can be packed with such complexes for example in order to be able to isolate cells which express the corresponding extracellular receptors is also feasible. Thus, the use of such complexes for detection purposes is also disclosed within the scope of the present invention.

A further subject matter of the present invention which is described here are fusion proteins which are suitable for the trimerization of [lacuna] as long as the recombinant fusion protein comprises at least one component A and at least one component B, component A comprising a protein or a protein segment with a biological function, in particular with a ligand function for antibodies or receptors, and component B comprising a trimerizing segment or a functional derivative of such a segment of a protein such as described above as constituent of the trimers according to the invention. Thus, all those recombinant fusion proteins are disclosed in accordance with the invention which are disclosed above as constituents of trimers according to the invention. The above disclosure—in connection with trimers according to the invention—regarding components A and B and for the construction of a recombinant fusion protein thus corresponds to the embodiments of a recombinant protein according to the invention per se. Thus, component B for a recombinant fusion protein according to the invention is typically a protein segment selected from the group consisting of the C1q protein family or the collectin family, or from the family of the collagenaceous proteins, component B of the recombinant fusion protein preferably exclusively comprising a trimerizing segment, but no trimer-oligomerizing structure or globular “head” domain. Typically, component B will thus comprise at least one amino acid sequence with the heptad pattern (abcdefg)ₙ which structurally forms a triple-helix-forming helix whose amino acids in positions a and d preferably have attached to them apolar side chains and thus enable the formation of the above-described superhelical structure, in this case as a triple helix composed of three helices. Such sequences of at least one heptad pattern, preferably at least two, can originate for example from one of the following proteins keratin, collagen, C1q, MBP, SP-A, SP-D, BC, CL43 or ACRP30. A functional derivative of such a segment of the abovementioned proteins may also be employed within the scope of the present invention, the above-selected definition of a functional derivative for component A analogously also applying to component B.

A further subject matter of the present invention is an inhibitor which is present in vitro in solution as hexamer (2×3) owing to the formation of a disulfide bridge (ApoFasL-060). This inhibitor is present in vivo—if appropriate in an oxidizing medium—in the form of a trimer or behaves in a trimer-like fashion and thus exerts inhibitory properties. ApoFasL-060 consists of an N-terminal flag sequence, a linker and a specific linker and the AAs 103 to 138 from hFasL and the AAs 139 to 281 (component A), likewise from hFasL (see FIG. 1). Analogously, an inhibitor of the ApoFasL-060 type as component A may also bear the corresponding binding segments of other TNF cytokines, for example OX40L, RANKL, TWEAK, Lta, Ltab2, LIGHT, CD27L, 41-BB, GITRL, APRIL, VEGI and BAFF or their segments or derivatives. The proteins CD40L, FasL, TRAIL, TNF (in particular TNF which binds to the receptor TNF-R2), CD30L and EDA and their segments and derivatives, in particular their respective human sequences, are very especially preferred.

The present invention is illustrated in greater detail by the figures which follow:

FIG. 1 shows the amino acid sequence of the FasL chimeras according to the invention (FasL-199, FasL-060 and FasL-267) in the one-letter code. The two protein chimeras FasL-199 and FasL-267 comprise a constituent of the protein ACRP30, a plasma protein which resembles structurally the complement factor C1q and which is produced by adipocytes. The native ACRP30 protein has a length of 247 amino acids, with a secretion signal sequence (AA 1 to 17) at the N terminus and a subsequent sequence of 27 amino acids (AA 18 to 44), which is responsible for the oligomerization of the protein. The subsequent segment (AA 45 to 110) of the native protein comprises 22 collagenaceous sequence repeats which, accordingly, form the coiled-coil domain. In the native state, this coiled-coil domain brings about the trimerization.

The protein chimera FasL-199 (control) was constructed with the aid of a PCR amplification and comprises the complete oligomerization domain of murine ACRP30 (mACRP30) (amino acids 18 to 110). In the direction of the C terminus, the FasL chimeric protein the trimerization domain of FasL (amino acids 139 to 281). Linker sequences are located between the N-terminal flag tag and the mACRP30 segment and between mACRP30 (component B) and the human hFasL segment (component A) (LQ).

The chimeric protein FasL-267 corresponds largely to the construct FasL-199, but has a deletion of the mACRP30 segment. It does not contain the oligomerization domain (amino acids 18 to 44) of ACRP30. The deletion mutant was generated from the EST clone AA673154 by PCR methods. The deletion of the amino acids 18 to 44 of mACRP30 causes the construct to exist in the form of a trimer, as demonstrated by the gel filtration experiments.

The chimeric protein FasL-060 has a flag sequence at the N terminus, followed by a linker (GPGQVQLQ), a specific linker which can form a disulfide bridge, the AA 103 to 138 of human FasL (hFasL) and, finally, as component A, the AA 139 to 281 of hFasL. Depending on whether the disulfide bridge is established, ApoFasL-060 behaves like a trimer or a hexamer.

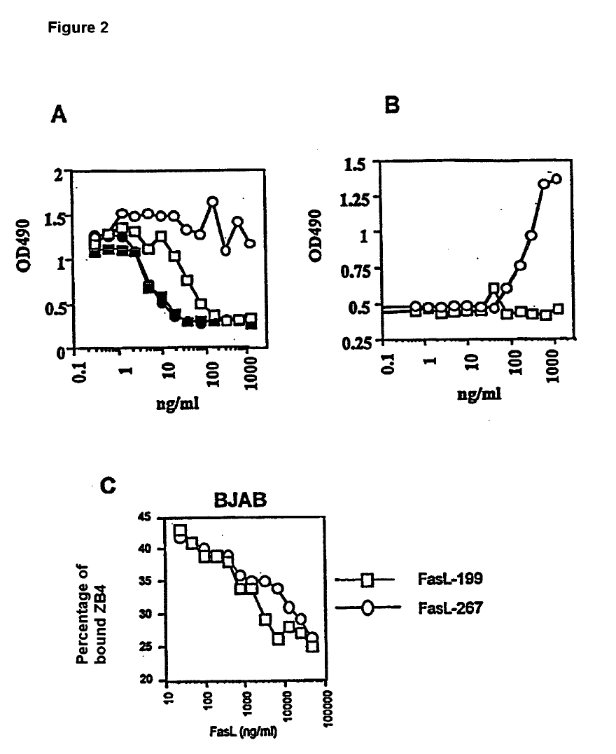

In FIG. 2A, FIG. 2 shows the activity of ApoFasL-060 and ApoFasL-267 in vitro with regard to the viability of Bjab cells. The absorbance at OD 490 nm is plotted on the y axis and the concentration of the fusion proteins added for the cytotoxicity test is plotted (logarithmically) on the x axis. The optical density at 490 nm is a measure of the viability of the cells (a high optical density corresponds to a low apoptotic activity of the substances added, and thus to high cell viability). The curves shown are curves for assays with ApoFasL-267 (o), ApoFasL-060 ( ), in each case without addition of crosslinking antibody, or in each case with addition of crosslinking antibody (●, ▪). FasL-267 alone is not cytotoxic for the cells, even at high concentrations (i.e. slight dilution), in contrast to FasL-267 with crosslinking antibody.

FIG. 2B shows the inhibitory activity of ApoFasL-267 (o) and ApoFasL-060 ( ) as shown in FIG. 2A. In order to be able to determine the inhibitory activity, oligomerized FasL at a concentration of 50 ng/ml was added in all experiments in order to trigger apoptosis.

FIG. 2C shows the results of affinity studies, in each case comparing the affinity of FasL-199 and FasL-267 to Bjab cells. ApoFasL-267 and FasL-199 compete with ZB4 antibodies for the binding to Fas on BJAB cells. The percentage of bound ZB4 (an anti-Fas antibody) is plotted versus the concentration of FasL-199 ( ) and FasL-267 (o), respectively. Within the inaccuracies of measurement, no difference was found with regard to the affinity of the two FasL ligands. It was thus demonstrated that the difference between the two ligands with regard to their cytotoxicity is not based on different binding affinities for the receptor.

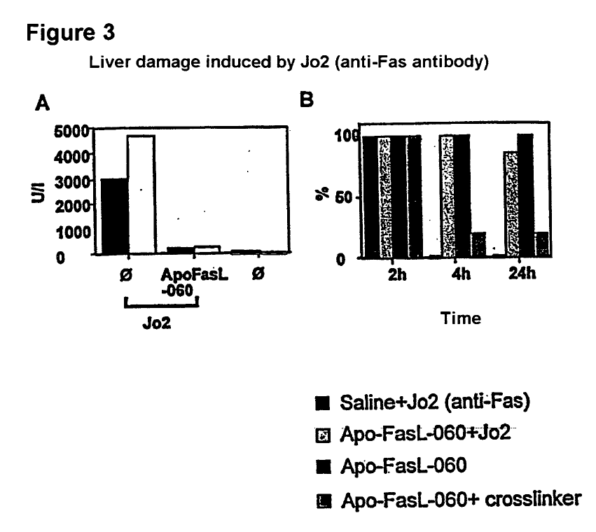

FIG. 3 shows the results of in-vivo experiments, namely the effect of ApoFasL-060 inhibition on hepatolysis as induced by agonistic anti-Fas antibodies J02. FIG. 3A shows the results of experiments in which mice were injected intravenously either with ApoFasL-060 (25 μg/mouse) or saline (control) before being injected intravenously with 5 μg of J02 antibody. The solid bars in FIG. 3A represent the serum titers (in U/ml) of ALT, while the open bars represent those of AST as the results of measurements four hours after iv injection. The plot on the right shows the result of the control experiment. FIG. 3B shows the survival rate of mice which have received 10 μg of J02 antibody, either after pretreatment with saline solution (solid bars) or with ApoFasL-060 (20 μg) (pale gray bars, in each case 2nd from left) or only with ApoFasL-060 (dark gray bars, in each case third from left) or with ApoFasL-060 and crosslinking antibody (mid-gray bar, in each case on the right), as a function of the time that has elapsed after the administration (2h, 4h, 24h). After 4h, none of the mice which have received exclusively agonistic J02 antibody (black bars) is still alive, while the inhibitor (pale gray bars) allows the survival of virtually all of the mice. Thus, ApoFasL-060 prevents the lethal action of the agonistic anti-Fas antibody J02 in mice. The crosslinking antibody, in turn, cancels the inhibitory effect of ApoFasL-060 (mid-gray bars).

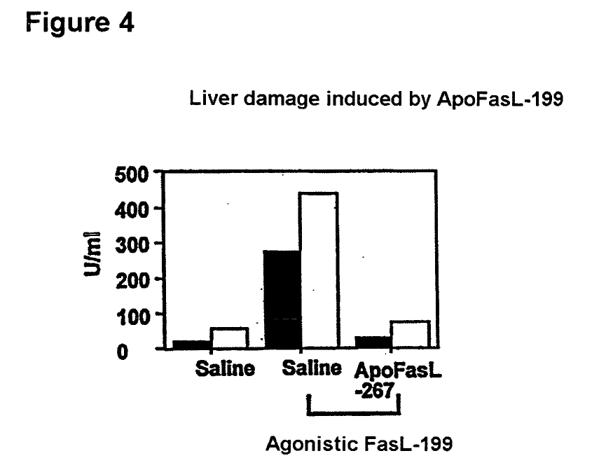

FIG. 4 shows the effect of ApoFasL-267 after liver damage induced by oligomerized FasL. FIG. 4 shows the results of experiments in which the mice were injected intravenously either with saline or with 25 μg of ApoFasL-267 before being injected intravenously with oligomerized FasL (FasL-199). The serum titers of ALT (solid bars) and AST (open bars) were analyzed after four hours had elapsed. Again, the bars represent the respective titers in U/ml. The plots in the middle and on the right in FIG. 4 show the results after the administration of agonistic, i.e. apoptosis-triggering, FasL, while the plot on the left represents the comparative experiment without administration of agonistic FasL. FIG. 4 thus not only shows the results of control experiments, but also the results of experiments with FasL-199 without protective ligand and of FasL-199 in combination with protective ligand FasL-267.

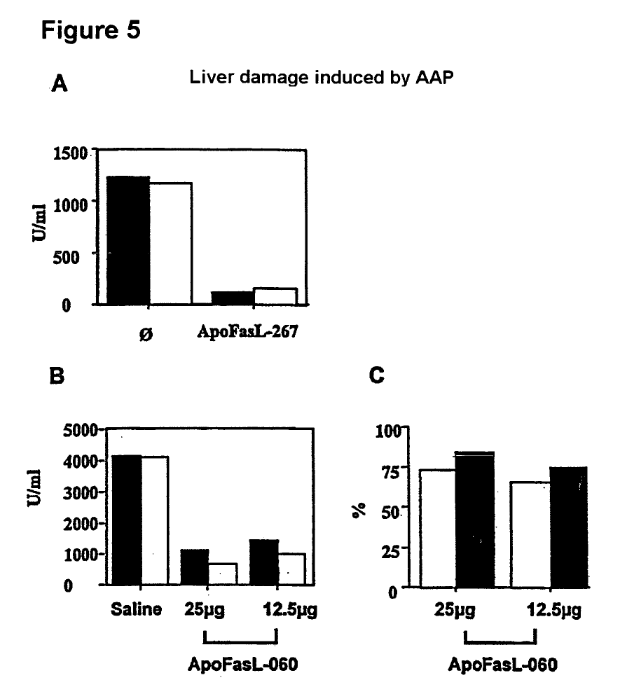

FIG. 5 shows the effect of soluble FasL in the case of AAP-induced hepatitis. The mice were injected intravenously either with ApoFasL-267 (FIG. 5A) or ApoFasL-060 (FIGS. 5B and 5C) before being injected intraperitoneally with AAP (300 mg/kg). The plots in FIGS. 5A and 5B are the titers (U/ml) of ALT (solid bars) and AST (open bars), measured in each case five hours after the injection. The plots on the left in FIGS. 5A and 5B correspond to the comparative experiments, while the plot on the right (FIG. 5A) and, in the case of FIG. 5B, the plot in the middle and on the right show the results after administration of the inhibitors according to the invention. FIG. 5C shows the relative decrease of the aminotransferase titers in comparison with mock-treated (control) animals (100%).

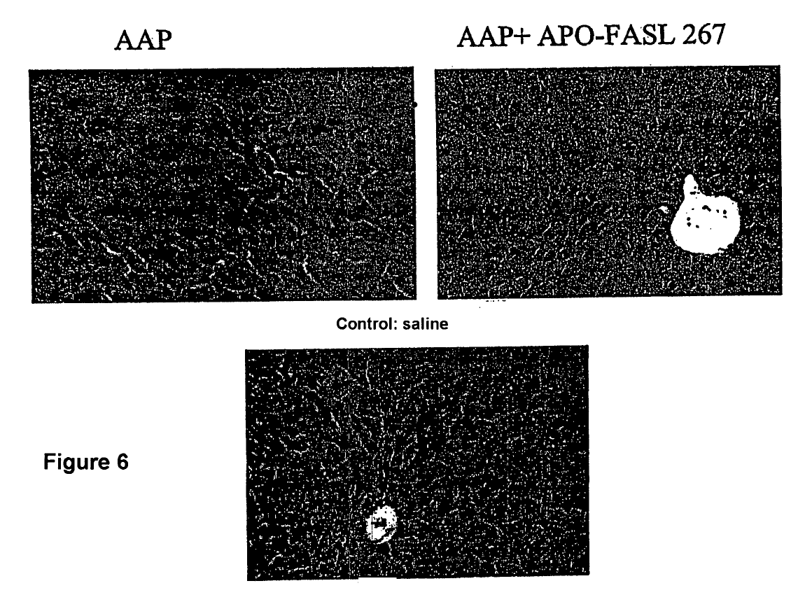

FIG. 6 shows the effect of ApoFasL-267 and ApoFasL-060 on an AAP-treated murine liver. The mice were treated as described above in FIG. 5. 24 hours after the induction of hepatitis, the livers were dissected and evaluated histologically. FIG. 6 contains three images of histological sections (addition of AAP, addition of AAP and ApoFasL-267 and, finally, a comparative set-up (control) after addition of saline). Treatment of the mice with ApoFasL-267 (or ApoFasL-060, not shown) prevents liver damage, as can be seen from a comparison with the histological section from the control animals. In contrast, the livers of exclusively AAP-treated animals show necrosis and apoptosis in the central venula region, sinusoidal inflammatory congestion of blood and vacuolized hepatocytes.

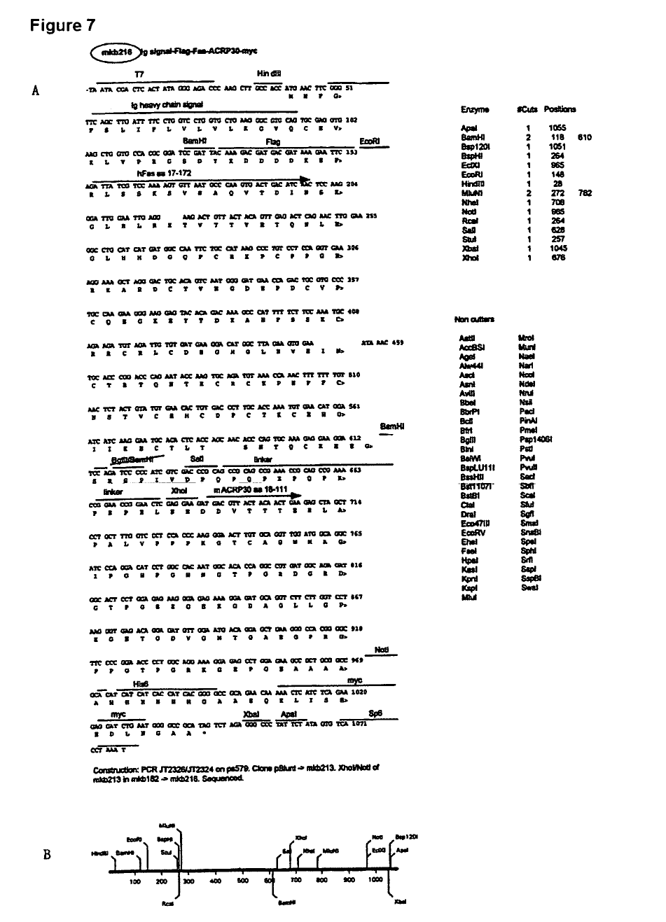

FIG. 7A shows the cDNA sequence and its derived amino acid sequence of the Fas chimera according to the invention, Fas-ACRP30 (MKB216). The construct comprises the amino acids 17 to 172 of the extracellular Fas domain (that is to say the Fas receptor), fused via a linker 14 amino acids in length to the complete oligomerization domain of murine ACRP30 (amino acids 18 to 110). At its amino terminus, the construct furthermore comprises, a signal sequence of the Ig heavy chain and a flag tag. Moreover, the restriction cleavage sites are shown in the figure.

FIG. 7B shows a restriction map, showing the cleavage sites of the construct of FIG. 7A.

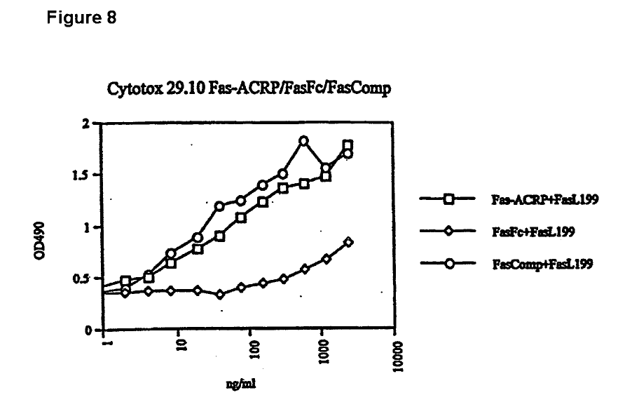

FIG. 8 documents the inhibitory action of Fas-ACRP30 in vitro against FasL-mediated apoptosis in A20 cells. The absorption at 490 nm (measure of cell survival; cf. also FIG. 2) is plotted on the y axis, while the concentration of the fusion protein in question is plotted on the x axis in ng/ml. The inhibitory effect of Fas-ACRP30 was compared with that of Fas-Fc, a dimeric form of Fas, and that of Fas-COMP, a pentameric form of Fas. The apoptosis-inducing agent used was the FasL chimera according to the invention, FasL-199. The Fas-ACRP30 construct inhibits FasL-induced apoptosis with an IC₅₀ value of 80 ng/ml. This value is comparable with the value of the pentameric Fas derivative Fas-COMP (35 ng/ml), while the IC₅₀ value for (dimeric) Fas-Fc is over 1 μg/ml.

The present invention is illustrated in greater detail by the use examples which follow:

The following experimental conditions (a) to (f) apply to the six use examples which follow, unless specified otherwise herein:

(a) Vector Constructions for the FasL, TRAIL, TNFα and CD40L Fusion Proteins:

The trimerization domain of FasL (AA 139-281) was amplified from human cDNA using the oligonucleotides JT398 (ACT GCA GGA AAA AAA GGA GCT G) and J290 (CAA CAT TCT CGG TGC CTG TAA C). The PCR product was ligated into pCRII (In Vitrogen) and the coding sequence, framed by the restriction cleavage sites PstI and EcoRI, comprising a DNA fragment encoding the hemagglutinin signal peptide, including six bases of the 5′-untranslated sequence (CAA AAC ATG GCT ATC ATC TAC CTC ATC CTC CTG TTC ACC GCT GTG CGG GGC) and the flag epitope (GAT TAC AAA GAC GAT GAC GAT AAA), the linker (GGA CCC GGA CAG GTG CAG), the restriction cleavage sites PstI, SalI, XhoI and BamHI, was then subcloned between the restriction cleavage sites HindIII and BamHI of a modified pCRIII vector (PS038, In-Vitrogen, NV Leek, The Netherlands) in which the bases 720-769 were deleted (PS 038).

The expression vector for FasL-199 was constructed as follows. Using the EST clone AA673154, a PCR amplification was first carried out with the aid of the oligonucleotides JT1147 (ACA ATG CAT GAA GAT GAC GTT ACT AC) and JT1148 (AGA CTG GAG AGC GGC TTC TCC AGG) The sequence encoding the amino acids 18 to 111 of the murine ACRP30, framed by the restriction cleavage sites NsiI and PstI, [lacuna] cloned into the PstI cleavage site of the vector encoding trimeric FasL (in such a way that the fused NsiI/PstI cleavage site was on the 5′ side of the coding sequence). The vector for expressing the fusion protein FasL-167 (with the AA 44-111 of mACRP30) was amplified with the aid of the alternative 5′-oligonucleotide JT1421 (AAA ATG CAT GCA GGC ATC CCA GGA C). The PCR product was ligated into a PCR “blunt” system, and the Nsi/PstI cassette was subcloned into the FasL-containing vector as described above. Other fusion proteins with alternative TNF cytokines in combination with ACRP30 were generated by substituting the respective sequence of FasL in the expression vector FasL-ACRP30 by the respective ligand sequence into the restriction cleavage sites PstI and EcoRI.

(b) Expression and Purification of the Recombinant Proteins:

HEK293 cells were transfected stably with the aid of the calcium phosphate method. After incubation for three days, the HEK293 cells were grown for two weeks in a selection medium comprising 800 μg/ml G418 (see also loc. cit.: Schneider et al., J. Exp. Med. 1998). These stably transfected clones were removed and distributed into 96-well plates containing selection media. The supernatants were analyzed for the presence of the recombinant protein with the aid of the anti-flag Western blot technique.

The stably transfected cells were grown for 10 to 14 days in 800 ml of a nonselective medium in flasks. The culture was centrifuged and the supernatant was filter-sterilized. Fusion proteins of FasL with murine ACRP30 (FasL-267 or FasL-199) were then purified as follows. The supernatants were treated with NaCl and CaCl₂ (final concentrations 150 mM and 2 mM, respectively), and the pH value was brought to 7.0 with aqueous hydrochloric acid/sodium hydroxide solution. Thereafter, the recombinant protein was applied to a 1 ml M2-agarose column (Sigma, Switzerland) (0.5 ml/min, 48 hours, 4° C.), and the column was washed with 10 volumes of TBS comprising 2 mM CaCl₂, finally eluted in TBS-EDTA (10 mM) (0.1 ml/min, 4° C.) or 50 mM citrate/NaOH (pH 2.5) (1 ml/min, 4° C.) and, if appropriate, neutralized with 0.2 volume of 1 M Tris-HC1 (pH 8). The buffer was exchanged for PBS in concentrators with a 30 kDA exclusion limit (Millipore). The concentration of purified proteins was determined by the bicinchonic acid method (Pierce Chemical Co., Rockford, Ill., USA) using bovine serum albumin as the standard, and the sample purity was determined by SDS-PAGE and Coomassie-Blue staining.

(c) Cells:

The human T-lymphoplastoma Jurkat cells, BJAB Burkitt lymphoma cells or Raji cells were grown in RPMI accompanied by 10% FCS. The human embryonic kidney cells 293 were cultured in a DMEM multi-substance mix F12 (1:1), supplemented with 2% FCS. All of the media comprised antibiotics (penicillin and streptomycin at in each case 5 μg/ml and neomycin at 10 μg/ml).

(d) Cytotoxicity assay:

The cytotoxicity assay was carried out essentially as described above by Schneider et al. (J. Biol. Chem. 272:18827-18833, 1997). Here, 50 000 cells were incubated for 16 hours in 100 μl of medium, the medium comprising the above-shown ligand concentrations in the presence or absence of 1 μg/ml M2 antibody. The cell survival rates were determined with the aid of PMS/MTS (phenanzine methosulfate 3-[4,5-dimethylthiazol-2-yl]-5-[3-carboxymethoxyphenyl]-2-[4-sulfophenyl]-2H-tetrazolium, salt) (Promega Corp., Madison, Wis.). The color was allowed to develop for the required period of time (typically 1-3 hours). The absorbance was measured at 490 nm. The optical density at 490 nm is a measure of cell viability (a high optical density corresponds to a low apoptotic effect of the added substances and thus to high cell viability).

(e) Treatment of the Mice

Female Balb/c mice (8 to 10 weeks old) were injected intravenously with the various constructs. The mice were bled after the periods of time stated, and the titers of the aminotransferases AST and ALT (aspartate aminotransferase and alanine aminotransferase) were subsequently quantified.

(f) Materials

The agarose-coupled anti-flag-M1 and anti-flag-M2 antibodies were obtained from Sigma (Buchs, Switzerland). The J02 antibodies were obtained from Pharmingen, and the cell culture reagents from Life Sciences (Basle, Switzerland).

(g) Binding Study

The affinity of ApoFasL-267 and FasL-199 was determined using a competition assay. To this end, the monoclonal ZB4 anti-Fas antibodies were labeled with 100 μCi (125I) using the iodo-Gen method (Pierce, Rockford, Ill.). This led to a specific activity of 1.5 μCi/μg protein. 1×105 Bjab cells were incubated for one hour at 37° C. with radio-labeled ZB4 and unlabeled competitors in serial dilution (100 μg/ml to 10 ng/ml). After three washes with cold PBS comprising 1% BSA, the radioactivity bound to the cells was determined using a γ-counter and expressed as percentage of bound cpm. All experiments were carried out in triplicate.

Furthermore, as regards the description of the methods employed for carrying out the use examples, Schneider et al. (J. Exp. Med., Vol. 187, No. 8, 1998, 1205-1213) and the publications cited therein as references are expressly referred to.

Use Example 1

A recombinant fusion protein (1, FasL-199) which comprised the amino acids 139 to 281 of hFasL (h: human) as component A and, as component B, a sequence 94 AA in length (AA 18 to 111 of mACRP30) N-terminally of amino acid 139 of component A was expressed. At the N terminus of the fusion protein (N-terminally of component B), there was additionally expressed a flag sequence with the amino acids DYKDDDDK and a linker sequence GPGQVQLQLH arranged between the flag tag and component B coupled on (see FIG. 1). Components A and B are separated by the linker sequence LQ.

For comparative experiments, a fusion protein (2, FasL-267) which comprised, at its N terminus, likewise the abovementioned flag sequence with the same linker sequence following C-terminally, and, in C-terminal arrangement, the amino acids 139 to 281 of hFasL was expressed. Fusion protein (1) differed from fusion protein (2) accordingly by a deletion encompassing the specific linker and the amino acids 103 to 138 of hFasL (FIG. 1).

The vectors for the fusion proteins (1) and (2) were constructed as described in the procedure above. The fusion proteins were expressed and purified as detailed in the method of (b).

The degree of multimerization or oligomerization of the purified fusion proteins (1 and 2) was determined by electron microscopy. The result was that FasL-199 as hexamer (2×3mer), and corresponding measurements for FasL-267 gave results for a trimer and for ApoFasL-060 likewise a hexamer.

Use Example 2

Inhibitory effect of ApoFasL-060 and ApoFasL-267 on Fas-mediated apoptosis in vitro.

Bjab-Burkitt lymphoma cells grown as described under (c) were removed and subjected to a cytotoxicity assay as described under (d). For this cell line, the assay was carried out in each case with increasing concentrations of trimerized fusion proteins ApoFasL-060 and ApoFasL-267 in the presence or absence of anti-flag M2 antibodies (Sigma, Buchs, Switzerland) (FIG. 2A) by determining the absorbance at OD 490 nm. The inhibitors applied, ApoFasL-060 and ApoFasL-267, are shown in FIG. 1 (see use example 1).

Both inhibitors reveal similar apoptosis-inducing properties when crosslinked with the antibody directed against the flag tag (FIG. 2A). ApoFasL-060 also induces apoptosis when no crosslinking antibodies are present, due to its aggregate structure. The results of use example 2 thus demonstrate that, while ApoFasL-060 and ApoFasL-267 are each capable of binding to the Fas receptor, they require oligomerization for transducing the death signal. The reduced cytotoxicity of the two inhibitors is not attributable to a reduced affinity for the Fas receptor since their affinity for oligomerized FasL (FasL-199) is not reduced.

In addition, Bjab cells were preincubated with increasing ApoFasL-267 concentrations, and FasL-199 was subsequently added to induce apoptosis (see FIG. 2B). It emerged that FasL-199-induced apoptosis can be prevented by ApoFasL-267. It can be seen from the series of measurements that ApoFasL-267 in the form of the trimeric molecule is capable of preventing apoptosis in vitro at an inhibitory concentration (IC) of 100 ng/ml. Thus, while ApoFasL-267 in trimeric form prevents apoptosis by blocking the receptor, aggregating ApoFasL-060 cannot prevent FasL-199-induced apoptosis in vitro since it itself has an apoptosis-inducing effect due to its structure.

### Use Example 3

In-Vivo Experiments after the Induction of Hepatolysis

A. Effect of ApoFasL-060

To this end, mice were injected with agonistic J02-anti-Fas antibodies which resulted in the deaths of the mice treated thus owing to fluminating hepatic failure. Hepatolysis was detected in these mice by the high aminotransferase (AST and ALT) serum titers. The mice were experimentally pretreated with ApoFasL-060 (1 mg/kg), thus protecting the animal after administration of J02 antibodies from the hepatic failure (hepatolysis) triggered by the latter (see FIG. 3A). As expected, the protective effect of ApoFasL-060 was dose-dependent. When 5 μg of J02 antibodies were administered, the AST and ALT titers observed corresponded to those of the control mice. At a dose of 10 μg of J02 antibodies, a predominantly protective effect was observed (approx. 90% reduction of AST and ALT titers). The result is thus that ApoFasL-060 per se is not toxic unless a crosslinking antibody (FIG. 3B) was employed, so that, again, the apoptosis-inhibiting effect is based on the binding to the Fas receptor without triggering the death signal.

The lethal dose of J02 antibodies (10 μg/per mouse, injected intravenously) led to death within four hours in all the mice studied (FIG. 3B). Pretreatment of these animals with ApoFasL-060 reduced the mortality rate drastically—the survival rate after 24 hours amounts to 86%. The inhibitor ApoFasL-060 (injected on its own) proves to be not toxic—after coinjection with crosslinking antibody, however, it has a highly toxic effect with a mortality rate of 80% after four hours.

B. Effect of ApoFasL-267

In this use example, the mice were injected either with saline (as control) or ApoFasL-267 prior to being injected with FasL-199. The respective AST and ALT serum titers in a hepatolytic analysis of the mice treated thus were subsequently assessed (see FIG. 4). The ApoFasL-267-pretreated mice are protected against hepatolysis as induced by the oligomeric FasL (FasL-199). In the animals employed as controls, FasL-induced hepatolysis can be observed very rapidly (within two hours) after the administration of FasL-199, showing aminotransferase titers which are increased by a factor of 10. Accordingly, pretreatment of the animals with ApoFasL-267 prevents liver damage of the animals as determined by the AST and ALT serum titers.

### Use Example 4

Inhibition of Acetaminophen (AAP)-Induced Hepatitis

Acetaminophen (AAP), a painkiller, is known to induce fulminating hepatic failure. The molecular mechanism is based on Fas-mediated apoptosis. The possibility of trimeric ApoFasL-267 and/or hexameric ApoFasL-060 protecting the liver cells from AAP-induced apoptosis was therefore investigated in the present use example. To this end, the mice were injected intraperitoneally with a sublethal dose of AAP (0.3 g/kg). Five hours later, any liver damage was determined by determining the ALT and AST serum titers. The ALT and AST titers were determined in an enzymatic assay in accordance with the IFCC (International Federation of Clinical Chemistry) Guidelines. The administration of ApoFasL-267 or ApoFasL-060 prevented an increase in the AST or ALT titers as brought about by AAP. In comparison with untreated mice, the aminotransferase titers at the time of observation in the mice which had been pretreated in accordance with the invention were markedly reduced (75 to 90%).

The protective effect of pretreatment with ApoFasL-060 proved to be dose-dependent (see FIGS. 5B and 5C). 25 μg of ApoFasL-060 (1 mg/kg) were administered, no AAP-induced hepatolysis was observed, when 12.5 μg were injected, the aminotransferase titers were slightly increased, i.e. slight liver damage occurred, and even lower doses, for example 6 μg/mouse, resulted in the loss of the protective effect. No difference was observed between these low doses and the control animals which had only been treated with saline. It can thus be stated that the inhibition observed takes the form of a competitive occupation of the binding sites on the receptor.

The AAP-induced liver damage was examined histologically (FIG. 6A). The following must be mentioned: necrosis and apoptosis in the central venula region, sinusoidal inflammatory congestion of blood, vacuolized hepatocytes. In contrast, as shown in FIG. 6B, no such symptoms of liver damage are discernible when a pretreatment with ApoFasL-267 (or ApoFasL-060, not shown) is carried out.

### Use Example 5

A. Construction of a Fas Receptor Chimera

To prepare an extremely effective inhibitor of FasL-mediated apoptosis, a fusion protein consisting of the extracellular domain of the Fas receptor (amino acids 17 to 172) and the complete oligomerization domain of murine ACRP30 (amino acids 18 to 110), which are linked via a linker 14 amino acids in length, was prepared (FIG. 7). The recombinant protein was analyzed by means of SDS-PAGE and had an apparent molecular weight of 55 kDa under reducing conditions and of 150 kDa under nonreducing conditions. It can thus be concluded that the construct MKB216 (hereinbelow referred to as Fas-ACRP30) occurs essentially in the form of a hexamer (2×3mer).

B. Inhibitory Effect of Fas-ACRP30 on FasL-Mediated Apoptosis

To confirm the inhibitory effect of the fusion protein Fas-ACRP30 according to the invention on FasL-mediated apoptosis in vitro, FasL-sensitive A20 cells were preincubated with increasing Fas-ACRP30 concentrations before oligomerized FasL was added. The construct FasL-199 according to the invention acted as particularly effective FasL oligomer in the present experiment. In this manner, the inhibitory effect of the construct Fas-ACRP30 according to the invention was compared with the effect of a dimeric form of Fas (Fas-Fc) and a pentameric form of Fas (Fas-COMP). As shown in FIG. 8, a Fas-ACRP30 concentration of 80 ng/ml can bring about a 50% reduction in FasL-mediated apoptosis. This value is markedly lower than that of the dimeric comparative construct Fas-Fc (IC₅₀>1 μg/ml). The inhibitory effect of Fas-COMP, which has an IC₅₀ value of 35 ng/ml, is comparable with that of Fas-ACRP30. These data thus confirm that Fas-ACRP30 is an effective inhibitor of FasL-mediated apoptosis.

1. A trimer of a recombinant fusion protein, characterized in that the recombinant fusion protein comprises at least one component A and at least one component B, component A comprising a protein or a protein segment with a biological function, in particular with a ligand function for antibodies, for soluble or membrane-bound signal molecules or for receptors or an antibody or antibody segment, and component B comprising a protein or a protein segment which trimerizes component A.

2. A trimer of a recombinant fusion protein as claimed in claim 1, characterized in that the components A of the recombinant fusion proteins in the trimer are identical or different.

3. A trimer of a recombinant fusion protein as claimed in claim 1, characterized in that component A of the recombinant fusion proteins is a peptide hormone, a growth factor, a cytokine, an interluekin, a receptor, a segment of these or a functional derivative of the abovementioned sequences.

4. A trimer of a recombinant fusion protein as claimed in any of claim 1, characterized in that component A of the recombinant fusion protein is a cytokine from the TNF cytokine family or a TNF cytokine receptor, a segment of such a TNF cytokine or receptor, or a functional derivative of a TNF cytokine or receptor or of a segment of such a TNF cytokine or receptor.

5. A trimer of a recombinant fusion protein as claimed in claim 1, characterized in that component A of the recombinant fusion protein is a TNF cytokine or a segment of a TNF cytokine selected from the group consisting of CD40L, FasL, TRAIL, TNF-α, CD30L, OX40L, RANKL, TWEAK, Lta, Ltab2, LIGHT, CD27L, 41-BB, GITRL, AP-R1L, EDA, VEGI and BAFF, or a functional derivative of the abovementioned sequences.

6. A trimer of a recombinant fusion protein as claimed in claim 1, characterized in that component B comprises a protein segment which comprises at least one heptad pattern of a collagen domain.

7. A trimer of a recombinant fusion protein as claimed in claim 1, characterized in that component B comprises a segment of the protein ACRP30, including the trimerization domain, without being capable of forming higher aggregates of trimers.

8. A trimer of a recombinant fusion protein as claimed in claim 1 characterized in that component B comprises an amino acid sequence as shown in FIG. 1 for the sequence of amino acid 44 to 111 of ACRP30, or a functional derivative of this sequence, where FIG. 1 is a constituent of the claim.

9. A trimer of a recombinant fusion protein as claimed in claim 1, characterized in that the recombinant fusion protein has the FasL-267 sequence as shown in FIG. 1, where FIG. 1 is a constituent of the present claim.

10. A trimer of a recombinant fusion protein as claimed in claim 1, characterized in that the recombinant fusion protein comprises a linker sequence between component A and component B.

11. A trimer of a recombinant fusion protein, as claimed in claim 1, characterized in that the linker sequence comprises the dipeptide LQ.

12. A trimer of a recombinant fusion protein as claimed in claim 1, characterized in that the recombinant fusion protein comprises a tag sequence, preferably an N-terminal tag sequence.

13. A trimer of a recombinant fusion protein, characterized in that at least two of the three recombinant fusion proteins present in the trimer have different components B.

14. The use of trimers as claimed in claim 1 for the preparation of a medicament.

15. The use of trimers as claimed in claim 1 for the preparation of a medicament for the treatment of viral hepatitis (HBV, HCV), alcohol-induced hepatitis, cholestatic hepatitis, Wilson's disease, autoimmune hepatitis, rejection in liver transplants, diseases due to hyperapoptotic reactions, degenerative diseases, in particular neurodegenerative diseases, inflammatory diseases, toxic epidermal necrolysis (TEN), multiple sclerosis, Hashimoto's thyroiditis, GvHD.

16. A recombinant fusion protein, characterized in that the recombinant fusion protein comprises a component A and a component B, component A being a protein or a protein segment with a biological function, in particular with a ligand function for antibodies or receptors or an antibody or antibody segment or for soluble or membrane-bound signal molecules, and component B comprising a trimerizing segment or a functional derivative of such a segment of a protein, in particular selected from the group consisting of the C1q protein family and the collectin family.

17. A DNA sequence, characterized in that the DNA sequence encodes a recombinant fusion protein as claimed in claim 1.

18. An expression vector, characterized in that the expression vector comprises a DNA sequence as claimed in claim 17.

19. A host cell, characterized in that the host cell is transfected with an expression vector as claimed in claim 18.
